(19) United States
(12) Patent Application Publication
(10) Pub. No.: US 2007/0269524 A1
(43) Pub. Date: Nov. 22, 2007

(54) Use of Idrocilamide for the Preparation of a Pharmaceutical Composition of Rosacea

(75) Inventors: Fabrizio Dolfi, Valbonne (FR); William Robert Pilgrim, Valbonne (FR)

Correspondence Address: OBLON, SPIVAK, MCCLELLAND MAIER & NEUSTADT, P.C., 1940 DUKE STREET, ALEXANDRIA, VA, 22314, US

(73) Assignee: Galderma Research & Development, S.N.C., Valbonne Sophia Antipolis (FR)

(21) Appl. No.: 10/580,251

(22) PCT Filed: Nov. 10, 2004
(86) PCT No.: PCT/FR04/02899

§ 371 Date: Apr. 5, 2007

(30) Foreign Application Priority Data
Nov. 21, 2003 (FR) 0313666

(51) Int. Cl. A61K 31/185; A61K 31/4164; A61K 9/50; A61P 17/00

(52) U.S. Cl. 424/490; 514/398; 514/576

(57) ABSTRACT

The invention relates to the use of idrocilamide for the preparation of novel pharmaceutical compositions, more particularly dermatological compositions, which can be used to treat rosacea.

The present invention relates to the field of treating rosacea. The invention is directed towards providing novel pharmaceutical compositions, more particularly dermatological compositions, which are useful for treating rosacea and which comprise idrocilamide as active agent.

Rosacea is a common, chronic and progressive inflammatory dermatitis associated with vascular instability. It mainly affects the central part of the face and is characterized by redness of the face or hot flushes, facial erythema, papules, pustules and telangiectasia. In serious cases, especially in men, the soft tissue of the nose may swell and produce a bulbous swelling known as rhinophyma.

Rosacea generally occurs between the ages of 25 and 70, and is much more common in people of fair complexion. It more particularly affects women, although this affection is generally more severe in men. Rosacea is chronic and lasts for years with periods of exacerbation and of remission.

Rosacea was originally called “acne rosacea” because its papules (points of slight raising of the skin) and its inflammatory pustules (pus scabs) greatly resemble those of common acne. In contrast with common acne, whose aetiology is based on abnormal keratinization, an increase in sebum production and also bacterial inflammation, the inflammation of rosacea is vascular in nature and is poorly understood. The result of this facial vascular anomaly is a permanent oedema of the dermis, which may be accompanied by an increased colonization with Demodex folliculorum, a mite usually found in the follicles of the face. This parasite might trigger inflammatory phenomena reflected by papules and pustules.

The pathogenesis of rosacea is poorly understood. Many factors may be involved without necessarily inducing this complaint. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

Rosacea develops in four stages, but passage through all the stages is not obligatory:
stage 1 of vasomotor flushes (at about 20 years old). The patients have sudden bursts of paroxystic redness of the face and neck, with a hot sensation, but with no systemic signs. After the attacks, the skin of the face returns to normal. These “flushes” are triggered by changes in temperature (occasionally leading to thermophobia), and the intake of hot drinks or alcohol;
stage 2 of erythemato-telangiectasia (at about 30 years old). The cheekbone areas are diffusely red. Dilated capillaries constituting standard acne rosacea are observed therein. In contrast with stage 1, the redness is permanent. Besides the cheeks, the chin and the middle of the forehead may be affected;
stage 3 of papulo-pustules (at about 40 years old). Papules and pustules a few millimetres in diameter develop on a background of erythema, without associated comedones. This dermatitis may be very extensive, occasionally up to the bald part of the scalp in men, but is absent from the area around the mouth and the eyes. The patients complain of sensitive skin, with subjective intolerance to the majority of topical products and greasy cosmetics;
stage 4 of rhinophyma (at about 50 years old or later). This late phase mainly affects men, in contrast with the other stages. The nose is increased in volume and diffusely red, and the follicular orifices are dilated. The skin gradually thickens.

The minor forms of rosacea may be treated with active agents such as anti-seborrhoeic agents and anti-infectious agents, for example benzoyl peroxide, retinoic acid or metronidazole (antiparasitic agent). As regards the most diffuse forms of the complaint, they respond well to general antibiotic therapy with cyclines. However, these treatments have unpleasant side effects for the patient, such as irritation or intolerance phenomena.

Furthermore, on account of the multi-factor aspect of rosacea, there are a huge number of treatments for this condition, but the search continues for an effective treatment that is without risk for the patient.

The Applicant has now demonstrated the advantageous properties of a compound belonging to the non-steroidal anti-inflammatory family (NSAIDs), idrocilamide, for treating rosacea.

NSAIDs are classified as a function of their chemical structure:
salicylic acid derivatives (for example aspirin, sulfasalazine, sodium salicylate, salsalate, diflunisal or olsalazine);
para-aminophenol derivatives (for example acetaminophen);
indole and indoleacetic acids (for example indomethacin, sulindac or etodolac);
arylacetic acids (for example tolmetin, diclofenac or ketorolac);
arylpropionic acids (for example ibuprofen, naproxen, ketoprofen, idrocilamide, fenoprofen or oxaprozin);
anthranilic acids (fenamates) (for example mefanamic acid or meclofenamic acid);
enolic acids (for example oxicams (piroxicam or tenoxicam) and pyrazolidiones (phenylbutazone or oxyphenthratazone));
alkanones (for example nabumetone).

NSAIDs are anti-inflammatory compounds known in the prior art for their analgesic and antipyretic properties. Idrocilamide, or N-(2-hydroxyethyl)cinnamide, is sold in particular by the Merck company in the pharmaceutical composition Sirilane, for its muscle-relaxant properties. Idrocilamide corresponds to the following formula:

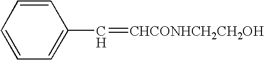

Moreover, patent application EP 0 270 316 describes the use of NSAIDs in topical compositions, in combination with 1-substituted imidazole, for treating acne. International patent application WO 02/074 290 discloses the use of certain NSAIDs in pharmaceutical preparations for treating rosacea.

However, it has never been proposed to use idrocilamide to treat rosacea. In the context of the present invention, it has now been found that idrocilamide has particularly advantageous properties in the treatment of rosacea, such as, especially, increased efficacy in particular in the case of individuals with fair or sensitive skin, a considerable dimunition of the side effects, probable efficacy in all the stages of rosacea and limitation of the phenomena of recurrence.

As indicated previously, the invention is directed towards offering a novel method for the pharmaceutical and preferentially dermatological treatment of rosacea, which consists in topically administering an effective amount of idrocilamide to an individual suffering from this condition.

Consequently, the invention relates more particularly to the use of idrocilamide for the preparation of a pharmaceutical composition and more particularly a dermatological composition, for topical application to the skin, for treating rosacea.

According to the present invention, the term “treating rosacea” means treating and/or preventing rosacea, at one or more of the stages described previously.

According to a first embodiment of the invention, the composition is for treating the first stage of rosacea.

According to a second embodiment of the invention, the composition is for treating the second stage of rosacea.

According to a third embodiment of the invention, the composition is for treating the third stage of rosacea.

According to a fourth embodiment of the invention, the composition is for treating the fourth stage of rosacea.

According to a first preferential embodiment, the composition contains 0.0001% to 20% of idrocilamide, and more preferentially 0.001% to 10% of idrocilamide (expressed as a weight percentage).

According to a second preferential embodiment, the composition contains 0.1% to 5% of idrocilamide (expressed as a weight percentage).

According to a third preferential embodiment, the composition in cream form contains about 5% of idrocilamide (expressed as a weight percentage).

Needless to say, besides the use of idrocilamide, the present invention relates to the use of derivatives thereof. The term “derivatives” means compounds that differ from idrocilamide by substitution, addition or removal of one or more chemical groups.

Advantageously, the compositions of the invention comprise, besides idrocilamide, at least one other therapeutic agent capable of increasing the efficacy of the treatment. Non-limiting examples of such agents that may be mentioned include antibiotics, antibacterial agents, antiviral agents, antiparasitic agents, antifungal agents, anaesthetics, analgesics, antiallergic agents, retinoids, free-radical scavengers, anti-priruginous agents, keratolytic agents, anti-seborrhoeic agents, antihistamines, sulfides, immunosuppressant products and antiproliferative agents.

According to one preferential embodiment, the composition of the present invention also contains metronidazole.

The term “metronidazole” especially means 1-(2-hydroxy-ethyl)-2-methyl-5-nitroimidazole, but also analogues and derivatives thereof, which are especially soluble in the formulation excipients that are suitable for the galenical form used.

The compositions of the invention may also comprise any additive usually used in the pharmaceutical or dermatological field that is compatible with idrocilamide. Mention may be made especially of sequestrants, antioxidants, sunscreens, preserving agents, for example DL-α-tocopherol, fillers, electrolytes, humectants, dyes, common mineral or organic acids or bases, fragrances, essential oils, cosmetic active agents, moisturizers, vitamins, essential fatty acids, sphingolipids, self-tanning compounds such as DHA, skin calmative and protective agents such as allantoin, pro-penetrating agents and gelling agents. Needless to say, a person skilled in the art will take care to select this or these optional additional compound(s), and/or the amount thereof, such that the advantageous properties of the composition according to the invention are not, or are not substantially, adversely affected.

These additives may be present in the composition in a proportion of from 0 to 20% by weight relative to the total weight of the composition.

Examples of sequestrants that may be mentioned include ethylenediaminetetraacetic acid (EDTA), and also derivatives or salts thereof, dihydroxyethylglycine, citric acid and tartaric acid, or mixtures thereof.

Examples of preserving agents that may be mentioned include benzalkonium chloride, phenoxyethanol, benzyl alcohol, diazolidinylurea and parabens, or mixtures thereof.

Examples of humectants that may be mentioned include glycerol and sorbitol.

The compositions of the invention may contain one or more pro-penetrating agents in preferential concentrations ranging from 0 to 20% and more preferentially ranging from 0.6% to 3% by weight relative to the total weight of the composition. Among the pro-penetrating agents that are preferentially used, without this list being limiting, are compounds such as propylene glycol, dipropylene glycol, propylene glycol dipelargonate, lauroglycol and ethoxydiglycol.

Advantageously, the compositions according to the invention may also contain one or more wetting liquid surfactants in preferential concentrations ranging from 0 to 10% and more preferentially ranging from 0.1% to 2%.

Among the wetting agents that are preferentially used, without this list being limiting, are compounds of the Poloxamer family and more particularly Poloxamer 124 and/or Poloxamer 182.

The compositions of the present invention may be in any galenical form normally used for topical application, especially in the form of aqueous, aqueous-alcoholic or oily solutions, dispersions of the lotion or serum type, aqueous, anhydrous or lipophilic gels, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream, gel or ointment type, or alternatively microemulsions, microcapsules, microparticles or vesicular dispersions of ionic and/or nonionic type.

Preferably, the creams may be formulated from a mixture of mineral oil or from a mixture of beeswax and of water, which emulsifies instantaneously, to which is added the idrocilamide, dissolved in a small amount of oil such as almond oil.

The ointments may be formulated by mixing a solution of idrocilamide in an oil such as almond oil in warmed paraffin, followed by leaving the mixture to cool.

As examples of compositions according to the invention, mention may be made of those comprising an active phase containing (expressed as weight percentages):
0 to 90%, preferentially 5% to 25% and especially 10% to 20% of water;
0 to 10%, preferentially 0 to 2% and especially 0 to 0.5% of wetting liquid surfactant;
0 to 20%, preferentially 0 to 10% and especially 2% to 5% of pro-penetrating agent;
0.0001% to 20% and preferentially 0.001% to 10% of idrocilamide;
   and an aqueous phase comprising a pH-independent gelling agent, and water.

The aqueous phase of a composition according to the invention in the form of an emulsion may comprise water, a floral water such as cornflower water or a natural spring or mineral water chosen, for example, from eau de Vittel, the waters of the Vichy basin, eau d'Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizieres, eau de Neyrac-les-Bains, eau de Lons-le-Saunier, les Eaux Bonnes, eau de Rochefort, eau de Saint Christau, eau des Fumades, eau de Tercis-les-Bains, eau d'Avène and eau d'Aix-les-Bains.

The said aqueous phase may be present in a content of between 10% and 90% by weight and preferably between 20% and 80% by weight relative to the total weight of the composition.

Non-limiting examples that may be mentioned include gelling agents of the polyacrylamide family such as the sodium acryloyldimethyltaurate copolymer/isohexa-decane/polysorbate-80 mixture sold under the name Simulgel 600 by the company SEPPIC, the polyacrylamide/C13-14 isoparaffin/laureth-7 mixture, for instance the product sold under the name Sepigel 305 by the company SEPPIC, the family of acrylic polymers coupled to hydrophobic chains, such as the PEG-150/decyl/SMDI copolymer sold under the name Aculyn 44 (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch sold under the name Structure Solanace, or mixtures thereof.

The preferred gelling agents are derived from the polyacrylamide family, such as Simulgel 600 or Sepigel 305 or mixtures thereof.

The gelling agent as described above may be used in preferential concentrations ranging from 0.1% to 15% and more preferentially ranging from 0.5% to 5%.

The gels may preferably be prepared by dispersing or dissolving idrocilamide in a suitable ratio in a gel of carbomer, poloxamer or cellulose-based type.

### EXAMPLE 1

### Compositions

In this example, various concrete formulations based on compounds according to the invention are illustrated.

TOPICAL ROUTE

| | | |
|---|---|---|
| (a) Ointment | | |
| Idrocilamide | 0.020 | g |
| Isopropyl myristate | 81.700 | g |
| Fluid petroleum jelly oil | 9.100 | g |
| Silica | 9.180 | g |
| (b) Ointment | | |
| Idrocilamide | 0.300 | g |
| White petroleum jelly codex | qs 100 | g |

| | |
|---|---|
| (c) Nonionic water-in-oil cream | |
| Idrocilamide | 0.100% |
| Mixture of emulsifying lanolin | 39.900% |
| alcohols, waxes and oils | |
| Methyl para-hydroxybenzoate | 0.075% |
| Propyl para-hydroxybenzoate | 0.075% |
| Sterile demineralized water | qs 100% |
| (d) Lotion | |
| Idrocilamide | 0.100% |
| Polyethylene glycol (PEG 400) | 69.900% |
| 95% Ethanol | 30.000% |
| (e) Hydrophobic ointment | |
| Idrocilamide | 0.300% |
| Isopropyl myristate | 36.400% |
| Silicone oil (“Rhodorsil 47 V 300” | 36.400% |
| sold by Rhone-Poulenc) | |
| Beeswax | 13.600% |
| Silicone oil (“Abil 300 000 cSt” | qs 100% |
| sold by Goldschmidt) | |

1. A pharmaceutical composition for treating rosacea comprising idrocilamide.

2. The composition according to claim 1, wherein the composition is for treating at least one stage of rosacea.

3. The composition according to claim 1, wherein the composition is for treating the first stage of rosacea.

4. The composition according to claim 1, wherein the composition is for treating the second stage of rosacea.

5. The composition according to claim 1, wherein the composition is for treating the third stage of rosacea.

6. The composition according to claim 1, wherein the composition is for treating the fourth stage of rosacea.

7. The composition according to claim 1, wherein the composition comprises from about 0.0001% to 20% by weight of idrocilamide.

8. The composition according to claim 1, wherein the composition further comprises an active agent selected from the group consisting of antibiotics, antibacterial agents, antiviral agents, antiparasitic agents, antifungal agents, anaesthetics, analgesics, antiallergic agents, retinoids, free-radical scavengers, anti-priruginous agents, keratolytic agents, anti-seborrhoeic agents, antihistamines, sulfides, immunosuppressant products and antiproliferative agents.

9. The composition according to claim 1, wherein the composition further comprises metronidazole.

10. The composition according to claim 1, wherein the composition further comprises an additive selected from the group consisting of sequestrants, antioxidants, sunscreens, preserving agents, fillers, electrolytes, humectants, dyes, common mineral or organic acids or bases, fragrances, essential oils, cosmetic active agents, moisturizers, vitamins, essential fatty acids, sphingolipids, self-tanning compounds, skin calmative and protective agents, pro-penetrating agents and gelling agents, and mixtures thereof.

11. The composition according to claim 1, wherein the composition further comprises one or more wetting liquid surfactants.

12. The composition according to claim 1, wherein the composition comprises from about 0.001% to 10% by weight of idrocilamide.

13. The composition according to claim 1, wherein the composition comprises from about 0. 1% to 5% by weight of idrocilamide.

14. The composition according to claim 1, wherein the composition is in the form of aqueous, aqueous-alcoholic or oily solutions, dispersions of the lotion or serum type, aqueous, anhydrous or lipophilic gels, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream, gel or ointment type, or alternatively ionic and/or nonionic microemulsions, microcapsules, microparticles or vesicular dispersions.

15. A topical dermatological composition for treating rosacea comprising idrocilamide.

16. A method for treating rosacea comprising applying topically to skin a pharmaceutical composition according to claim 1.
